(19) United States
(12) Patent Application Publication
(10) Pub. No.: US 2026/0256894 A1
(43) Pub. Date: Sep. 3, 2026

(54) METHOD OF TREATING CID BY PHE-IAP, IAP-TRP, AND PHE-IAP-TRP COMPLEXES

(71) Applicant: Biplab Kumar Malo, Reading, MA (US)

(72) Inventor: Biplab Kumar Malo, Reading, MA (US)

(21) Appl. No.: 19/068,828

(22) Filed: Mar. 3, 2025

(51) Int. Cl. A61K 38/46; A61K 9/00; A61K 31/198; A61K 31/405

(52) U.S. Cl. CPC A61K 38/465; A61K 9/006; A61K 31/198; A61K 31/405; C12Y 301/03001

(57) ABSTRACT

A method of treating and preventing disease includes administering a Phe-IAP, IAP-Trp, and or Phe-IAP-Trp complex, individually or in any combination, to a subject in an amount effective to normalize bioavailability of IAP, Phenylalanine, and Tryptophan in a subject's GI tract, blood, brain or any place in the body to prevent excessive production of tyramine, catecholamines, serotonin, melatonin, kynurenine, and indole-3-lactate and/or to reduce plaque and stone formation in the gastrointestinal tract, blood vessels, muscles, brain, or any other parts of the body. The administered composition is a tablet, capsule, gel or liquid, and is ingestible, injectable, or topically applied. The method can prevent or cure any disease that is associated with IAP, Phenylalanine, Tryptophan, and plaque.

### BACKGROUND OF THE INVENTION

The present invention relates to a method of treating or preventing diseases associated with bioavailable levels of intestinal alkaline phosphatase (IAP), phenylalanine (Phe), and tryptophan (Trp), and, more particularly, to using novel Phe-IAP, IAP-Trp, and Phe-IAP-Trp complex as the therapeutical biologics.

Neurotransmitters like catecholamines (CA) and serotonin play a significant role for maintaining homeostasis of the human body (Mittal et al, 2017). Elevated catecholamine is known to cause heart palpitation, tachycardia, panic disorder, anxiety 242, headache, and sweating 240. Imbalance of catecholamines is associated with gastrointestinal disorder. Optimal dopamine production is essential for normal function of the brain, and protection against the development of neurological disorders. (Hamamah et al, 2022). Imbalanced phenylalanine 214 would develop imbalanced production of catecholamines such as dopamine 222, norepinephrine 224, and epinephrine 226. Imbalanced tryptophan 216 would cause imbalanced production of serotonin 230 and melatonin 234. This would cause disruption of normal neural activity in the brain, and subsequently development of neurological and psychological disorders. Exogenous manipulation of these catecholamines (CA) serum concentration can help the disease progression associated with CA (Mittal et al, 2017).

Phenylalanine (Phe) and Tryptophan (Trp) are both essential amino acids. Both Phe and Trp need to be taken as protein in a diet. IAP is a natural enzyme produced in the gastrointestinal (GI) tract (Malo et al, 2010).

L-phenylalanine is a precursor amino acid of L-tyrosine for endogenous production. (Matthews, D). L-tyrosine is a large neutral amino acid that is the precursor of tyramine, L-dopa, and all catecholamines (Mittal et al, 2017), such as the neurotransmitters dopamine, norepinephrine, and epinephrine. Tyrosine is not an essential amino acid. Tyrosine hydroxylation is the rate limiting step for the synthesis of the major catecholamines including norepinephrine and dopamine (Marriott, B 1994). Decarboxylation of L-tyrosine produces Tyramine (Burns, C and Kidron, A 2022). See also Min, K., et al. (2019). A perspective on the biotechnological applications of the versatile tyrosinase. Bioresource technology, 289, 121730.

Tyramine is a vasoactive amine that promotes blood pressure elevation 240 and can result in pain. It leads to cerebral vasoconstriction and subsequent vasodilation which causes migraines 242 in some people (Costa, M. R., and Gloria, M.B.A. Migraine and Diet. Encyclopedia of Food Science and nutrition (second edition), 2003). “Ingestion of large amounts of tyramine, especially combined with monoamine oxidase inhibitors (MAOIs), can precipitate a hypertensive crisis” (Burns, C and Kidron, A 2022). Increased Phenylalanine 214 can cause increased L-tyrosine 212 which produces increased tyramine 210 and L-dopa 220. Increased L-dopa 220 produces increased dopamine 222, norepinephrine 224, 234, and epinephrine 226. Elevated tyramine 210, norepinephrine 224, 234, and epinephrine 226 causes hypertension, palpitation, tachycardia, tremor, and sweating 240. See also Gillman, P. K. (2018). A reassessment of the safety profile of monoamine oxidase inhibitors: elucidating tired old tyramine myths. Journal of Neural Transmission, 125(11), 1707-1717. 2% lidocaine with 1:100 K epinephrine is used every day in dental offices.

L-tryptophan is the precursor for 5-hydroxy-tryptophan (5-HTP). 5-HTP is the precursor for serotonin (5-HT). Increased Trp 216 would cause increased 5-HTP 228, kynurenine 232, indole 218, and other metabolites. Increased level of 5-HTP would produce increased serotonin 230 and melatonin 236, 234.

Some metabolites from L-phenylalanine and L-tryptophan are also pro inflammatory agents.

Chronic inflammatory diseases (CID) include Gastrointestinal diseases, Mental health conditions, Metabolic diseases, Cardiovascular diseases, atopic dermatitis, Autoimmune diseases (e.g., rheumatoid arthritis, lupus, psoriasis, and ankylosing spondylitis), Lung diseases (e.g., asthma and chronic obstructive pulmonary disease (COPD)), malignancy (such as colorectal cancer, endometrial cancer, and melanoma), chronic kidney disease, and liver disease.

Gastrointestinal diseases include dysbiosis, enterocolitis, Crohn's disease, ulcerative colitis, and irritable bowel syndrome (IBS). Imbalance of catecholamines is associated with gastrointestinal disorders. Some factors that negatively affect the immune-modulatory role of serotonin could result in different diseases in the GI tract including Crohn's, colitis, celiac disease and diverticulitis (Shaw et al., 2010). High serotonin levels resulted in enhanced biofilm formation and pathogenicity of the organism in vitro (Knecht et al, 2016). Increased level of tryptophan metabolism is associated with the activity of inflammatory bowel diseases (Nikolaus et al, 2017), and Trp deficiency could contribute to development of IBD (Nikolaus S et al, 2017). Exogenous administration of serotonin enhanced the production of pro-inflammatory cytokines, increased biofilm formation on mouse intestine, and worsened the pathological conditions (Mittal et el., 2017).

Mental health conditions include neurological and psychological disorders such as Autism spectrum disorder (ASD), attention deficit hyperactivity disorder (ADHD), depression, anxiety, panic disorder, schizophrenia, dyskinesia, dementia, sleep disorder 242, mania, and bipolar. The gut microbiota contain intrinsic enzymatic activity that helps production of catecholamine and serotonin synthesis from the available L-tyrosine and L-tryptophan, respectively (Mittal et al, 2017). Tryptophan catabolites generated by the gut microbiota are important signaling molecules among the microbiota and host-microbial crosstalk and might contribute to homeostasis (Roager, H. M. and Licht, T. R, 2018). Optimal dopamine production is essential for the normal function of the brain and protection against the development of neurological disorders. (Hamamah et al, 2022). The neurocircuitry in the gut-brain axis involves neurotransmitters (Cickovski et al, 2023). Therefore, dysbiosis could be a new therapeutic target and prognostic tool for the treatment of patients with major depressive disorder (M. Gao, et al., Association analysis of gut microbiota and efficacy of SSRIs antidepressants in patients with major depressive disorder, Journal of Affective Disorders, Volume 330, 2023, Pages 40-47, ISSN 0165-0327, doi: 10.1016/j.jad.2023.02.143). See also Gabriele, S., Sacco, R., & Persico, A. M. (2014). Blood serotonin levels in autism spectrum disorder: a systematic review and meta-analysis. European Neuropsychopharmacology, 24(6), 919-929.

Metabolic diseases or conditions include intestinal alkaline phosphatase deficiency (IAPD), Type 1 Diabetes (T1DM), Type 2 Diabetes (T2DM), Obesity 244, and phenylketonuria 208. U.S. Pat. Nos. 7,048,914 B2, and 7,858,085 B2 were issued to Zoltan Kiss to treat T1 D and T2D with alkaline phosphatase. IAPD leads chronic inflammation 246 to Type 1 and Type 2 Diabetes 244. Intestinal deficiency of IAP is defined as a concentration less than 65 unit/gm stool (Malo M, 2015). U.S. Pat. No. 10,781,470 B2 was issued to diagnose and treat incipient diabetes to Dr. MS Malo. At IAPD level, lipopolysaccharide (LPS) and lipoteichoic acid (LTA) concentrations start to rise in the gut and the integrity of the intestinal wall is disrupted. LPS and LTA are endotoxins. In Type 2 Diabetes, LPS and LTA affect the function of insulin (Malo J, 2022). In Type 1 Diabetes, they affect the production of insulin (Dr. M S. Malo, Boston Diabetes conference, 2023). Epinephrine and nor-epinephrine play a major role in absorption of nutrients from GI tract. Epinephrine causes increased absorption of glucose. Alpha receptor is the predominant receptor that governs the absorption of nutrients (Mittal et al, 2017). Exogenous administration of epinephrine causes significant hyperglycemia in rats. Combination of thyroid hormone and epinephrine causes even greater hyperglycemia (Mittal et al., 2017).

It is known that L-phenylalanine is a specific inhibitor for intestinal alkaline phosphatase (Malo, M 2015). It is also known that tissue nonspecific alkaline phosphates (TNAP) have a significant effect in teeth and bone. The buildup of phenylalanine 214 also causes phenylketonuria (PKU) 208, where Phe cannot be converted to L-tyrosine (Winn et al, 2018). When excessive amounts of phenylalanine 214 build up in the blood of the brain, it blocks other neutral amino acid transfer to the brain.

Chronic inflammatory diseases 246 are associated with degradation of tryptophan through kynurenine pathway 232 (Harris et al, 2024) and indole-3-lactate is associated with cerebral infarction (CI) 248. Obesity is also associated with systemic inflammation 246 with the altered Trp metabolic pathway (Cussotto S et al, 2020).

Cardiovascular diseases (CVD) include atherosclerosis, high blood pressure, tachycardia, heart palpitation 240, migraine 242, myocardial infarction (MI), cerebral infarction (CI) 248, seizure, and stroke. The novel factors involved in cardiovascular inflammatory diseases are the downstream metabolites of Trp. Tryptophan levels were inversely correlated with cardiovascular diseases while the metabolites kynurenine and serotonin 234 were positively correlated. (Teunis et al, 2023). Increased kynurenine 232, KYN/Trp ratio, indole-3-lactate 238 and serotonin 230, 234 are associated with fatal CVD (Teunis et al, 2023). High serotonin 230, 234 level is associated with higher peripheral artery disease (PAD), and high PAD is associated with myocardial infarction, and cerebral infarction 248 or stroke (Teunis et al, 2023). Kynurenine has been associated with atherosclerosis and cardiometabolic disease such as T2DM, and Obesity (Cussotto, S et al, 2020; Teunis et al, 2023). Both kynurenine (KYN) and microbiota mediated indole routes of Trp metabolism altered with high KYN/Trp ratio, and lower 5-HT (serotonin) and indole. Tryptophan catabolism by the kynurenine pathway (KP) 232 is associated with systemic inflammation 246 and cardiovascular disease in the patient with chronic kidney diseases (Konje V et al, 2021): “KP metabolites have the potential to direct therapy to patients at risk of CVD”. Tryptophan degradation might affect the systemic immunity in chronic inflammatory diseases (Harris, D et al, 2024). IAP knockout (KO) mice suffer from metabolic endotoxemia and systemic inflammation (Kaliannan et al. 2013; Dr. MS Malo, Obesity and Diabetes Conference 2023, Boston, MA).

Autoimmune diseases: serotonin (5-HT) receptors are found on lymphocytes, monocytes, macrophages, and dendritic cells (Cloez-Tayarani and Change, 2007). Serotonin is produced by macrophage and T-cells (Began and Blakely 2013). Tyrosine phosphorylation is a key means of signal transduction in the immune system that initiate signals from receptors, integrins, and cytokine receptors. By phosphorylation and de-phosphorylation of tyrosine multicellular organism communicate and express gene, cellular morphology, cellular proliferation, differentiation and death. Protein tyrosine phosphatase (PTPs) de-phosphorylate and protein tyrosine kinase (PTKs) phosphorylate Tyrosine. PTPs and PTKs keep the immune system balanced (Kiratikanon et al 2022).

Some of the Tryptophan is also catabolized by gut microbiota and produces metabolites such as tryptamine and indole (Roager H. M. and Licht, T. R., 2018). Catabolism of Trp also produces N-formylkynurenine (Teunis et al, 2023). Tryptophan catabolites generated by the gut microbiota are important signaling molecules among the microbiota, and host-microbial crosstalk and might contribute to homeostasis (Roager, H. M. and Licht, T. R, 2018). Increased levels of tryptophan metabolism are associated with the activity of inflammatory bowel diseases: a high level of Trp degradation has been observed in patients with IBD and Trp deficiency could contribute to development of IBD (Nikolaus S et al, 2017).

The gut microbiota contains intrinsic enzymatic activity that helps production of catecholamine and serotonin synthesis from the available L-tyrosine, and L-tryptophan respectively (Mittal et al, 2017). 95% Serotonin is produced in the gut (Terry, N. and Margolis, K). The enteric nervous system (ENS) governs the GI function. The interaction between the ENS and the central nervous system (CNS) is referred to as ‘gut-brain axis’ (Mittal et al, 2017). The gut-brain axis involves the Vagus nerve (Appleton, J 2018). The neurocircuitry in gut-brain axis involves neurotransmitters (Cickovski et al, 2023). It is now well established that L-tyrosine is intrinsically involved in the gut-brain axis (Mittal et al, 2017) as an ascending signal from gut to the brain, central nervous system (CNS) via enteric nervous system (ENS). Therefore, dysbiosis could be a new therapeutic target and prognostic tool for the treatment of patients with major depressive disorder 242 (Gao et al, 2023).

From the above, one can also postulate that imbalanced amounts of Phe and Trp results in imbalanced catecholamines and serotonin in the brain that causes headache 240, especially during hormonal changes in the body.

As can be seen, there is a need for a means of balancing the amounts of Phe and Trp in a subject.

### SUMMARY OF THE INVENTION

In one aspect of the present invention, a method of treating and preventing IAP-, Phe-, and Trp-associated diseases comprises administering to a subject Phe-IAP, IAP-Trp, and Phe-IAP-Trp complexes, individually or in any combination, effective to normalize bioavailability of Phenylalanine, Tryptophan and/or IAP in any part of the body. It could be administered by mouth or by injection subcutaneously in the muscle, transdermal patch, intravenously in the blood, directly to spinal fluid in the brain, topically as gel to the skin or in the vagina, or by nasal-oral spray inhalation or implantation.

In another aspect of the present invention, a composition for use in the method of treating and preventing Phe, Trp, and IAP associated diseases comprises the amount of Phe-IAP, IAP-Trp, and/or Phe-IAP-Trp individually or in any combination effective to bring bioavailability of Phenylalanine, Tryptophan, and/or IAP to a normal range of concentration in the subject's oral cavity, nasal cavity, gastrointestinal tract, vagina, blood, muscle, brain or any parts of the body.

The method described herein may cure or prevent any disease that is associated with Phenylalanine, Tryptophan, and/or IAP anywhere in the body.

IAP stores both Phe and Trp by catching, capturing, and forming as Phe-IAP, IAP-Trp, and/or Phe-IAP-Trp complexes. The complexes may have different forms and structure such as Phe-IAP-Phe in the presence of Phe only or Trp-IAP-Trp in presence of Trp only. The presence of the amino acids and IAP would dictate the structure in nature. IAP also releases both Phe and Trp from the stored complexes via dilution. Thus, IAP modulates the bioavailability of Phe and Trp to a normal range of concentration by storing and releasing, as the body demands. Both Phe and Trp are essential amino acids which are precursors for production of neurotransmitters such as Catecholamines, Serotonin, and other complex biological agents. Therefore, Phe-IAP, IAP-Trp, and Phe-IAP-Trp are directly involved in in chronic diseases (CID) such as metabolic disease, Gastrointestinal diseases, mental health disorders, and CVD. Low IAP 202 would cause high tryptophan 216 in GI tract resulting in high serotonin 230, 234, kynurenine 232, and indole-3-lactate 238. Low IAP would also cause high L-phenylalanine 214 resulting in high tyramine 210 and increased L-dopa 220. Therefore, IAP is the key regulator for production of neurotransmitters and metabolites produced from Phe and Trp such as Tyramine, Dopamine, Norepinephrine, Epinephrine, Serotonin, Melatonin, Kynurenine, Tryptamine, and Indole. Most diseases associated with these neurotransmitters and metabolites, thus, could be prevented or treated with administering Phe-IAP, IAP-Trp, and Phe-IAP-Trp complexes alone, or in any combination depending on the condition or the disease.

These and other features, aspects and advantages of the present invention will become better understood with reference to the following drawings, description, and claims.

### BRIEF DESCRIPTION OF THE DRAWINGS

FIG. 1 is a flow chart of a method of administration of a drug according to an embodiment of the present invention;

FIG. 2 is a schematic view of the effect of substance deficiencies on a patient; and

FIG. 3 is a photographic view illustrating experimental results of applying to biofilm-containing saliva a complex of phenylalanine, intestinal alkaline phosphatase, and tryptophan (Phe-IAP-Trp complex) according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a method of treating and preventing various diseases by administering to a subject a composition selected from the group consisting of: Phe-IAP complex, IAP-Trp complex, Phe-IAP-Trp complex, and any combination thereof.

As used herein, the term “subject” may refer to a human or an animal, including pets such as dogs and cats and barnyard animals such as pigs and cows, etc.

The diseases or conditions that may be treated by the present method may be associated with chronic inflammatory disease (CID) and/or plaque formation. Plaque associated diseases or conditions are dental caries, gingivitis, periodontitis, oral-pharyngeal lesion, IAPD, colorectal cancer (CRC), endometrial cancer, vaginitis, urinary tract infection (UTI), prostate cancer, gallbladder and kidney stones, atherosclerosis, endocarditis, dementia, and plaque formation on the surface of or in the fluid of the GI tract, veins, arteries, lungs, heart, kidneys, liver, pancreas, brain, skin, or anywhere in the body. Dementia may be related to plaque formation associated with P-tau protein, Fusobacterium nucleatum, Porphyromonas gingivalis, and other bacteria.

The treatment may be effective to maintain normal production of catecholamines, tyramine, and serotonin in the body to prevent or treat cardiovascular diseases. The treatment may be effective to stop excessive production of tyramine, catecholamines, kynurenine, and serotonin to prevent or treat cardiovascular diseases. The treatment may be effective to maintain production of dopamine and serotonin to prevent or treat neurological and psychological conditions. The treatment may be effective to prevent excessive production of dopamine and serotonin to treat neurological and psychological mental health conditions. The treatment may be effective to stabilize production of catecholamines and serotonin in the gut to prevent or treat gastrointestinal diseases. The treatment may be effective to reduce excess amounts of phenylalanine and/or tryptophan in the gut and brain to prevent or treat gastrointestinal diseases. The treatment may be effective to stabilize the amounts of IAP, Phe, and Trp in the body, especially in the gut, to prevent or treat metabolic diseases or conditions.

The treatment may be effective to stop or reduce attachment of biofilm and formation of plaque and stone anywhere in the body, especially in the oral-pharynx cavity, the gastrointestinal tract, the vagina, the endometrium, the arteries and veins, the heart, the lungs, the liver, the kidney, the gallbladder, the pancreas, and the brain.

The treatment may be provided as a tablet, capsule, injection, spray, transdermal application, implantation, or IV administration depending of the disease condition to protect all the body's systems.

The complex may be taken as a food supplement, and it can also be produced endogenously, provided Phenylalanine is bioavailable for endogenous production of L-tyrosine and Tryptophan is bioavailable for endogenous production of 5-HTP.

The complex may be applied to a non-biological material as a coating. The coating may be used in medical devices or instruments to maintain sterility.

I have discovered that IAP has additional functions besides breaking down Phosphodiester bond and preventing attachment of LTA and LPS. IAP catches, captures and stores the essential amino acids Phe and Trp, forming Phe-IAP, IAP-Trp, and/or Phe-IAP-Trp complexes. Each amino acid and IAP becomes bioavailable when diluted. It is noteworthy that IAP is not the same as the Phe-IAP, IAP-Trp, and Phe-IAP-Trp complexes. IAP alone is an active enzyme; however, IAP is inactive in the complex. Phe alone is different from Phe-IAP and Trp alone is different from IAP-Trp complex.

IAP is the key modulator of Phe and Trp bioavailability and balances Catecholamine and Serotonin production to maintain the homeostasis of the body. Therefore, it is believed that administering a Phe-IAP, IAP-Trp, and/or Phe-IAP-Trp complex alone or in any combination would prevent or cure chronic inflammatory diseases (CID) associated with Phe, Trp, and/or IAP, solving a problem at the initial stage of the disease process.

Phe, Trp, and IAP interact in the GI tract. When IAP interacts simultaneously with Phe and Trp, they form a Phe-IAP, IAP-Trp and/or Phe-IAP-Trp complex; and IAP becomes functional when diluted. Thus, it could be concluded that excessive consuming of Phe and/or Trp could be problematic in absence of IAP, or in IAP-deficient (IAPD) gut. It would cause an increased level of LPS, LTA, Phe, and Trp in the gut, making the condition extremely harmful for the body and making the body susceptible to previously described diseases. The inflammatory pathway and the autoimmune system get activated as the body's defense system in this condition.

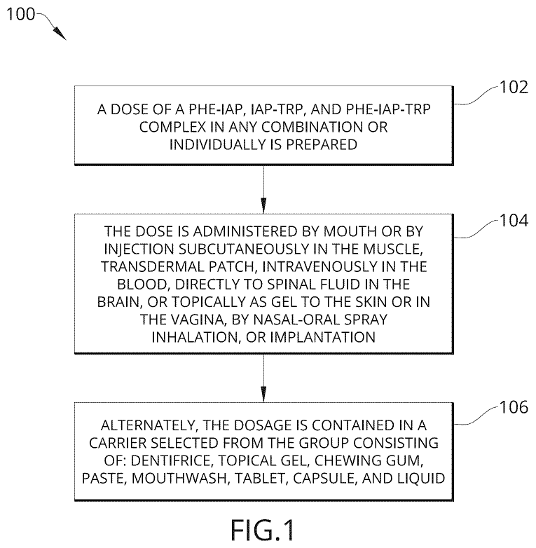

Referring to FIGS. 1-3, FIG. 1 is a flowchart of a method 100 of preventing, treating, or curing disease by administering a complex to a subject. The complex of Phe-IAP. IAP-Trp, and/or Phe-IAP-Trp may be prepared 102 synthetically in the lab or be extracted from natural resources and administered 104 in a variety of ways. The complex may be administered topically, transdermally, orally, intranasally, vaginally, subcutaneously, intravenously, intrathecally, or by implantation. The Phe-IAP, IAP-Trp, and/or Phe-IAP-Trp complex may be contained in a carrier selected from the group consisting of a dentifrice, nano particles, a gel, a chewing gum, a paste, a mouthwash, a tablet, a capsule, a liquid capsule, and a liquid 106. The Phe-IAP, IAP-Trp, and/or Phe-IAP-Trp complex could be produced in a vector such as yeast, algae, bacteria, or virus and delivered with the inscribed method. They could also be delivered as mRNA capsules for endogenous production.

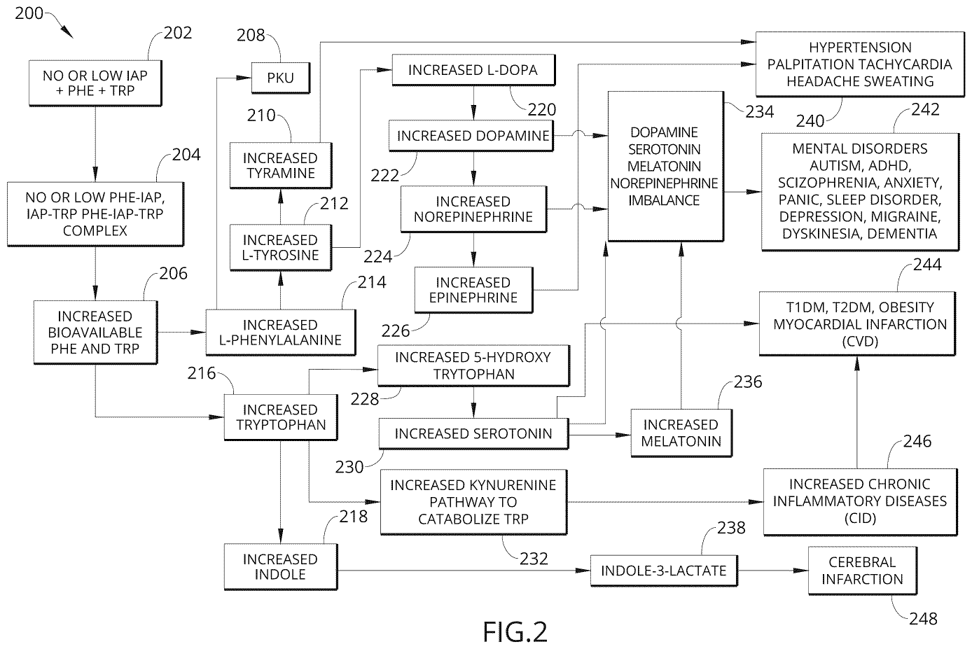

FIG. 2 is a flow chart illustrating the progression 200 of a disease that may occur due to no or low IAP 202 level in the gut, and increased bioavailability of Phe and Trp 206. When the subject has no or low IAP (IAPD) 202, a smaller amount of Phe-IAP, IAP-Trp, and/or Phe-IAP-Trp 204 will be formed, increasing catecholamines and serotonin in the body. Accordingly ingestion of the essential amino acids Phe and Trp (independent of complexes with IAP) would abruptly cause increased bioavailable Phe and Trp 206 in the gut, resulting in excessive and abrupt production of neurotransmitters and metabolites which are harmful for the body. The steps of disease progression 200 are discussed in more detail in the Background.

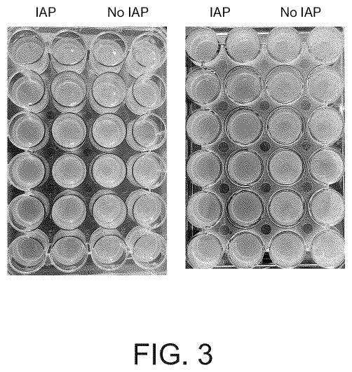

FIG. 3 illustrates experimental results, showing that attachment of biofilm from oral saliva was reduced or lost in the presence of a Phe-IAP-Trp complex having an IAP concentration of 55 unit/ml.

I started experimenting to investigate if Phe really destroys IAP and found that the Phe forms a Phe-IAP complex where IAP is non-functional. Phe-IAP complex was diluted and investigated to determine if the IAP function comes back. Indeed, IAP function came back when the complex was diluted.

I wanted to see if Trp interacts with IAP in the same way as Phe. I found that Trp also binds to IAP and inactivates IAP forming a Trp-IAP complex; IAP becomes functional when diluted.

Then, known quantities of both Phe and Trp were mixed in a solution and a known quantity of IAP was added.

In this example, much less IAP was functional than in complexes of Phe or Trp alone. The mixture formed a Phe-IAP-Trp complex. When the complex was diluted, the concentration of IAP increased, indicating IAP was non-functional in the complex and recovered functionality upon dilution. The presence of Phe after dilution of the complexes was detected using the Ferric Chloride test, and the presence of Trp after dilution of the complexes was detected using the Hopkins-Cole test. Those two tests confirm the bioavailability of Phe and Trp after dilution of the complexes.

EXAMPLE 1: A solution having a known IAP concentration of 155 U/ml was mixed with a solution of Phe. The bioactive IAP and Phe-IAP complex were measured using our patented STAP-Test (stool alkaline phosphatase test) and Phe-IAP complex. The concentration of IAP decreased as the concentration of Phe increased. However, upon dilution of Phe-IAP complex, the IAP became functional and bioavailable.

pNPP refers to para-nitro-phenyl-phosphate, a substrate that is essential for the STAP-Test solution. IAP cleaves the phosphate bond from the pNPP, forming pNP and P. The concentration of IAP is measured based on the rate of reaction, converting pNPP to pNP and P by IAP.

TABLE 1

| | | | | Diluted |
|---|---|---|---|---|
| | | | | Phe-IAP |
| | For measurement | Measured IAP | | complex |
| For measurement | of IAP function in | in Phe-IAP | | In pNPP |
| of IAP function in | pNPP solution with | complex | | ONLY |
| pNPP solution, | Phe | Bioavailable | Phe-IAP | Solution |
| dilution | (no dilution) | IAP | complex | (Dilution) |
| pNPP ONLY | pNPP + Phe | IAP coc. In | Known IAP- | Total IAP |
| | solution | presence of | Bioavailable | conc. U/ml |
| | | Phe | IAP | in pNPP |
| | | | | ONLY |
| 1 ml 10 mM pNPP | 10 mM PHE in | 65.89 U/ml | (155-65.89) | 166 U/ml |
| | 1 ml 10 mM pNPP | | U/ml = | |
| | | | 89.11 U/ml | |
| 1 ml 10 mM pNPP | 10 mM PHE in | 67.71 U/ml | 87.29 U/ml | 162 U/ml |
| | 1 ml 10 mM pNPP | | | |
| 1 ml 10 mM pNPP | 30 mM PHE in | 16.02 U/ml | 138.98 U/ml | 140 U/ml |
| | 1 ml 10 mM pNPP | | | |
| 1 ml 10 mM pNPP | 150 mM PHE in | 18.75 U/ml | 136.25 U/ml | 153 U/ml |
| | 1 ml 10 mM pNPP | | | |

EXAMPLE 2: A solution having a known IAP concentration of 32.5 U/ml was exposed to solutions of Phe having different concentrations. The average bioactive IAP was measured for each mixture. The concentration of bioactive IAP was observed to decrease as the concentration of Phe increased.

TABLE 2

| | | IAP Conc. | IAP Conc. | IAP Conc. |
|---|---|---|---|---|
| | | Unit/ml in | Unit/ml in | Unit/ml in |
| | Amount | 25 mM | 50 mM | 100 mM |
| | Used | PHE and | PHE and | PHE and |
| | Micro | 10 mM | 10 mM | 10 mM |
| Dilution | Liter | pNPP | pNPP | pNPP |
| 100 X D IAP | 20 | 10.48 | 6.9 | 4.7 |
| 100 X D IAP | 20 | 10.06 | 7.3 | 4.5 |
| 10 X D IAP | 20 | 11.81 | 6.78 | 3.2 |
| 10 X D IAP | 20 | 11.80 | 6.84 | 2.41 |
| 1 X D IAP | 5 | 7.91 | 4.9 | 3.52 |
| 1 X D IAP | 5 | X | 4.75 | 3.39 |
| 1 X D IAP | 10 | X | 8.1 | 3.5 |
| 1 X D IAP | 10 | X | 7.5 | 3.67 |
| AVERAGE | | 10.41 | 6.63 | 3.61 |
| Bioavailable IAP | | | | |
| Known IAP − | | 32.5 − 10.41 | 32.5 − 6.63 | 32.5 − 3.61 |
| Available | | | | |
| IAP | | | | |
| Phe-IAP complex | | 22.09 | 25.87 | 28.89 |

EXAMPLE 3: A solution having a known IAP concentration of 42.85 U/ml was exposed to solutions of Phe having different concentrations. The average bioactive IAP was measured for each mixture. The background concentration of the solution (a solution that is used to dilute IAP) was also measured to make sure that it does not impact the result. The concentration of bioactive IAP decreased as the concentration of Phe increased. Background solution is the solution of the pNPP and the substrate (Phe in this case) without the addition of IAP. Some substrates have effects on OD (optical density) which needs to be corrected if significant. In this example it is not significant, maximum 0.010 u/ml, and lowest 0.0025 u/ml.

TABLE 3

| | | Confirming | IAP Conc. | IAP Conc. | IAP Conc. |
|---|---|---|---|---|---|
| | | IAP Conc. | U/ml | U/ml | U/ml |
| Dilution | | U/ml in 10 | In 25 mM | In 50 mM | In 100 mM |
| Factor of | Amount of | mM pNPP | Phe and | Phe and | Phe and |
| IAP | IAP used | only | 10 mM pNPP | 10 mM pNPP | 10 mM pNPP |
| 100 X D IAP | 20 micro L | 43.533 | 13.0129 | 8.4799 | 5.6109 |
| 100 X D IAP | 20 micro L | 43.698 | 12.3523 | 8.8531 | 6.5203 |
| 10 X D IAP | 10 micro L | 43.538 | 9.911 | 7.402 | X |
| 10 X D IAP | 10 micro L | 42.471 | 10.215 | | X |
| 10 X D IAP | 20 micro L | X | 11.227 | 7.77513 | 4.74579 |
| 10 X D IAP | 20 micro L | X | 11.537 | | 4.88122 |
| 1 X D IAP | 5 micro L | X | X | 8.712 | 4.60925 |
| 1 X D IAP | 10 micro L | X | X | X | 4.27314 |
| AVERAGE | | 43.31 | 11.38 | 8.24 | 5.11 |
| Background | 20 micro L | 0.010 U/ml | 0.011 U/ml | 0.0025 U/ml | 0.005 U/ml |
| of pNPP - | | | | | |
| PHE solution | | | | | |
| | | | 42.85 − 11.38 | 42.85 − 8.24 | 42.85 − 5.11 |
| Phe-IAP | | | 31.47 | 34.61 | 37.74 |
| complex | | | | | |
| U/ml | | | | | |

The Phe-IAP complex increases proportional to both the IAP concentration and Phe concentration. IAP captures and collects Phe proportionally.

EXAMPLE 4: A solution having a known concentration of IAP was combined with a solution of Trp to form a IAP-Trp complex. The bioactive IAP concentration decreased as the Trp concentration increased. IAP becomes bioavailable when IAP-Trp complex diluted; confirming the existence of the IAP-Trp complex.

TABLE 4

| | | Measured |
|---|---|---|
| | Measured IAP | IAP-Trp complex |
| | conc., | conc., Unit/ml |
| Known IAP conc. = | Unit/ml | (known conc. − |
| 9.90 Unit/ml | (Bioavailable) | bioavailable conc.) |
| IAP Conc. 5 mM Trp in | 4.56 | 5.34 |
| 10 mM pNPP | | |
| IAP Conc. In 10 mM Trp in | 2.99 | 6.91 |
| 10 mM pNPP | | |
| AP Conc. In 15 mM Trp in | 2.16 | 7.74 |
| 10 mM pNPP | | |
| Dilution of IAP-Trp in | 9.016 | 0.88 |
| 10 mM pNPP | | |

| Known IAP conc. = | IAP concentration, | |
|---|---|---|
| 230 Unit/ml | Unit/ml | Difference |
| IAP Conc. In 5 mM Trp in | 152 | 78 |
| 10 mM pNPP | | |
| IAP Conc. 15 mM Trp in | 66 | 164 |
| 10 mM pNPP | | |
| IAP Conc. 25 mM Trp in | 32 | 198 |
| 10 mM pNPP | | |
| IAP Conc. 25 mM IAP-Trp | 212 | 18 |
| Dilution | | |

EXAMPLE 5: IAP appears to have a greater affinity for Trp than Phe. When IAP was added to a mixture of Phe and Trp, much less IAP was functional, indicating Phe and Trp have synergistic effects forming a Phe-IAP-Trp complex: more complex is formed than that of each complex with Phe or Trp individually. When the complex was diluted, more IAP was functional, confirming the existence of the Phe-IAP-Trp complex.

TABLE 5

| | | Ave. | | |
|---|---|---|---|---|
| Known | IAP | IAP | | |
| IAP conc. = | Conc. | conc. | Complex, | Utilization |
| 502.5 U/ml | U/ml | U/ml | U/ml | of IAP |
| 10 mM pNPP | 493 | 502.5 | | |
| 10 mM pNPP | 512 | | | |
| | | | Phe-IAP | |
| 25 mM Phe | 112.23 | 112.23 | 390.27 | 77.66% |
| | | | IAP-Trp | |
| 25 mM Trp | 41.80 | 40.8 | 461.7 | 91.88% |
| 25 mM Trp | 39.87 | | | |
| | | | Phe-IAP-Trp | |
| 25 mM Phe-IAP-Trp | 27.56 | 26.3 | 476.2 | 94.77% |
| 25 mM Phe-IAP-Trp | 25.10 | | | |
| Dilution of 25 mM Phe- | 225.71 | 225.71 | 276.79 | 55.08% |
| IAP-Trp in 10 mM pNPP | | | | |

It should be understood, of course, that the foregoing relates to exemplary embodiments of the invention and that modifications may be made without departing from the spirit and scope of the invention as set forth in the following claims.

What is claimed is:

1. A therapeutic composition comprising a complex selected from the group consisting of: phenylalanine and intestinal alkaline phosphatase (Phe-IAP complex); intestinal alkaline phosphatase and tryptophan (IAP-Trp complex); phenylalanine, intestinal alkaline phosphatase, and tryptophan (Phe-IAP-Trp complex); and any combination thereof.

2. The therapeutic composition of claim 1, further comprising a carrier selected from the group consisting of: dentifrice, nano particles, a topical gel, a chewing gum, a paste, a mouthwash, a tablet, a capsule, a gel capsule, a liquid, a liquid capsule, a biological vector, and an mRNA capsule.

3. The therapeutic composition of claim 1, wherein the composition is provided in a formulation selected from the group consisting of: an injectable liquid, a tablet, a capsule, a gel, a spray, a pill, a pellet, a liquid-containing capsule, a gel-containing capsule, a powder, a solution, an emulsion, a drop, a suppository, an aerosol, a suspension, a delayed release formulation, and a controlled-release formulation.

4. A method of treating or preventing a disease associated with phenylalanine (Phe), tryptophan (Trp), intestinal alkaline phosphatase, and/or plaque, comprising:
administering the composition of claim 1 to a subject in an amount effective to achieve within the subject a bioavailable level within a selected range of an amino acid and enzyme selected from the group consisting of: intestinal alkaline phosphatase, Phenylalanine, Tryptophan, and any combination thereof.

5. The method of claim 4, wherein the step of administering comprises ingesting the composition, and wherein the composition is a tablet or a capsule containing an amount of Phe-IAP, IAP-Trp, and/or Phe-IAP-Trp effective to prevent or treat intestinal alkaline phosphate deficiency (IAPD).

6. The method of claim 4, wherein the step of administering comprises ingesting the composition, and wherein the composition is a tablet or a capsule containing an amount of Phe-IAP, IAP-Trp, and/or Phe-IAP-Trp effective to prevent or treat diabetes.

7. The method of claim 4, wherein the step of administering comprises ingesting or injecting the composition, and wherein the composition is a tablet, a capsule, or a liquid containing an amount of Phe-IAP, IAP-Trp, and/or Phe-IAP-Trp effective to prevent or treat phenylketonuria (PKU) by maintaining a balance of catecholamines and serotonin in the subject.

8. The method of claim 4, wherein the step of administering comprises ingesting the composition, and wherein the composition is a tablet or a capsule containing an amount of Phe-IAP, IAP-Trp, and/or Phe-IAP-Trp effective to prevent or treat mental health disorders by maintaining a balance of catecholamines and serotonin in the subject.

9. The method of claim 4, wherein the composition is a liquid containing an amount of Phe-IAP, IAP-Trp, and/or Phe-IAP-Trp effective to prevent dementia associated with plaque formation by breaking down lipoteichoic acid (LTA), lipopolysaccharide (LPS), and P-tau protein in blood circulation, spinal fluid, and brain of the subject.

10. The method of claim 4, wherein the composition is a liquid or a gel containing an amount of Phe-IAP, IAP-Trp, and/or Phe-IAP-Trp effective to prevent cardiovascular diseases associated with plaque formation by breaking down lipoteichoic acid (LTA), lipopolysaccharide (LPS), and P-tau protein in blood circulation of the subject.

11. The method of claim 4, wherein the step of administering comprises ingesting or injecting the composition, and wherein the composition is a tablet, a capsule, or a liquid containing an amount of Phe-IAP, IAP-Trp, and/or Phe-IAP-Trp effective to prevent or treat headache, migraine, and chronic pain by maintaining a balance of catecholamines and serotonin in the subject.

12. The method of claim 4, wherein the disease is a chronic inflammatory disease selected from the group consisting of: a gastrointestinal disease, a mental health condition, a metabolic disease, a cardiovascular disease, atopic dermatitis, an autoimmune disease, a lung disease, a malignancy, a chronic kidney disease, and a liver disease.

13. The method of claim 4, wherein the disease is selected from the group consisting of dental caries, gingivitis, periodontitis, oral-pharyngeal lesion, intestinal alkaline phosphatase deficiency (IAPD), colorectal cancer (CRC), endometrial cancer, vaginitis, urinary tract infection (UTI), prostate cancer, gallbladder stones, kidney stones, atherosclerosis, endocarditis, dementia, and plaque formation.

14. The method of claim 4, wherein the step of administering is performed by mouth.

15. The method of claim 4, wherein the step of administering is performed by injection subcutaneously in muscle.

16. The method of claim 4, wherein the step of administering is performed intravenously in blood.

17. The method of claim 4, wherein the step of administering is performed directly to spinal fluid.

18. The method of claim 4, wherein the step of administering is performed topically.

19. The method of claim 4, wherein the step of administering is performed vaginally.

20. The method of claim 4, wherein the step of administering is performed intranasally.
